(19) United States
(12) Patent Application Publication
(10) Pub. No.: US 2011/0041386 A1
(43) Pub. Date: Feb. 24, 2011

(54) Extraction From Suspensions

(76) Inventors: Daniel Fleischer, Oakland, CA (US); Andrew Thompson, Oakland, CA (US); Marko Jukic, San Francisco, CA (US); Guido Radaelli, Oakland, CA (US); Shaina Jackson, Berkeley, CA (US)

Correspondence Address: CARR & FERRELL LLP, 120 CONSTITUTION DRIVE, MENLO PARK, CA, 94025, US

(21) Appl. No.: 12/544,079

(22) Filed: Aug. 19, 2009

(51) Int. Cl. C10L 1/18; C11B 1/00; C12M 1/00

(52) U.S. Cl. 44/308; 554/1; 44/307; 554/8; 210/321.6

(57) ABSTRACT

A suspension may include an aqueous liquid and suspended particles. The particles may include a nonpolar and/or hydrophobic substance (e.g., a lipid) substantially contained within polar and/or hydrophilic exterior layers. A method for extracting the suspended lipids may include adding a nonpolar solvent to the suspension and disrupting the exterior layers to expose the lipids to the nonpolar solvent. In some cases, particles may also include interior hydrophilic portions (e.g., intracellular water), which may be exposed to the aqueous liquid via disruption of the exteriors. The mixture may be accelerated to segregate the mixture into first and second products. The first product may have a majority of the nonpolar and/or hydrophobic substances. The second product may have a majority of the polar substances.

### BACKGROUND

1. Technical Field

The present invention relates generally to extracting substances from suspensions.

2. Description of Related Art

A suspension may include a suspended phase and a liquid. In some applications, separation of a suspended phase (or portion thereof) from the liquid may be desired. A portion of a suspended phase might dissolve in a solvent, but may be immiscible with the liquid, and may be enclosed within a layer having chemical compatibility with the liquid but not the solvent. Extraction of the immiscible portion of the suspended phase may be hindered by properties of the liquid.

FIG. 1 illustrates a suspension of particles. In this example, suspension 100 includes an aqueous liquid 110 and suspended particles 120. Suspended particles 120 may include a substantially polar exterior 130 and an interior that may include a substantially non-polar portion 140 (e.g., a lipid or nonpolar portions thereof). Polar exterior 130 may be a cell wall, a micelle wall, a bilayer, an organization or arrangement of polar moieties, and the like. Such a configuration may inhibit extraction of non-polar portion 140 from the suspension.

Extraction (e.g., of lipids, proteins, carbohydrates, and the like) may be challenging with aqueous suspensions. The effectiveness of many separation processes may be reduced when a suspension includes a substantial amount (e.g., over 10%, 20%, 60%, 80%, or even over 90%) of water. Removing water (e.g., by evaporation) may require substantial energy.

### SUMMARY OF THE INVENTION

A suspension may include particles suspended in an aqueous liquid. The particles may include a nonpolar and/or hydrophobic substance (e.g., a lipid) substantially contained within polar exterior layers. A method for extracting the suspended lipids may include adding a nonpolar solvent to the suspension and disrupting the exterior layers to expose the lipids to the nonpolar solvent. This mixture may be segregated into first and second products, the first product having a higher amount of the lipids than the second product.

A suspended phase may be a solid, a liquid, a composite, or another phase. In some cases, suspended phases may include small particles (e.g., less than 100 microns, less than 10 microns, less than 1 micron, or even less than 100 nm). A suspended phase may include a photosynthetic organism (or a plurality thereof), such as algae, diatoms, and the like.

An aqueous liquid may include water, such as natural or synthetic seawater. A concentration of the aqueous liquid may be greater than 10%, greater than 50%, or even greater than 90% by mass of the suspension. Exterior layers may be cell walls, layers formed by hydrophilic moieties, and/or other layers that inhibit interaction between at least a portion of the interiors and an added solvent. An added solvent may be an alkane, such as hexane.

Disrupting the exterior layers may include agitating the suspension and solvent. Agitating may include stirring, blending, ultrasonicating, and other mechanically induced forces. Disrupting may include lysing. Disrupting may include adding a chemical that “unlocks” or otherwise breaches the exteriors. Disrupting may include heating (e.g., to above 80 degrees Celsius). In some cases, pressure over a heated mixture may be controlled, for example to maintain the liquid and/or solvent in a liquid state.

Disrupted particles may be segregated. Segregating may include agglomerating particles to form larger volumes of similar moiety or polarity, which may be more effectively acted upon by segregation forces. Segregating may include allowing the mixture to settle into separate segregation products. Segregating may include centrifuging (e.g., at 200, 500, or even 2000 RCF). In some cases, a hydrophobic substance such as a lipid may be dissolved by a nonpolar solvent, and a segregation product may include this solution. Portions of disrupted particles may agglomerate, particularly when disruption of exterior layers allows for interaction between interior regions having similar polarity that might otherwise be separated by the (opposite polarity) exteriors.

Liquids, lipids, proteins, and/or carbohydrates may be extracted. In some cases, substances may be extracted from photosynthetic organisms, including single celled organisms such as algae and diatoms. In some embodiments, lipids are extracted from members of the genus Nannochloropsis.

A biofuel may be fabricated from extracted substances (e.g., using transesterification to produce methyl-esters from lipids, or fermentation of carbohydrates to produce alcohols, or partial combustion of carbonaceous substances to form syngas). A substance (e.g., non-lipid biomass) may be fabricated using various methods, and may have a mean particle sizes associated with the organisms from which the substance was extracted. In some cases, fine particulate biomass results from a residual non-lipid biomass extracted from algae.

A method may comprise providing a suspension in a pressure vessel. The suspension may include suspended cells in natural or synthetic seawater. The suspended cells may include interior lipids surrounded by hydrophilic exteriors. Hexane may be added to the suspension to form a mixture. The mixture may be heated to between 20 and 200 degrees Celsius, in some cases under controlled pressure that maintains the hexane in a liquid state. The mixture may be agitated (e.g., stirred at 300 rpm). The agitated mixture may be accelerated in a centrifuge (e.g., at 2300 RCF) to segregate the mixture into a first product of predominantly hexane and lipids, and a second product that is predominantly the natural or synthetic seawater, and may also include intracellular water. In some cases, a third product includes a majority of the non-lipid biomass and/or residual solids.

### BRIEF DESCRIPTION OF THE DRAWINGS

FIG. 1 illustrates a suspension of particles.

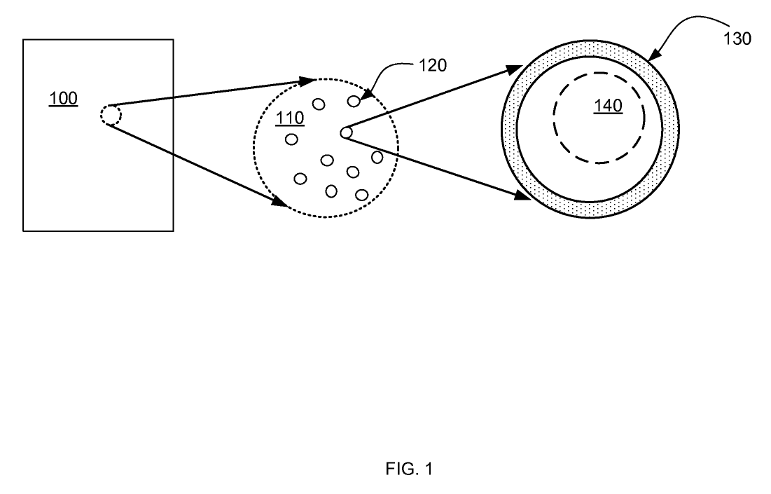

FIG. 2 illustrates disruption of an exterior layer, according to some embodiments.

FIG. 3 illustrates a method according to some embodiments.

FIG. 4 illustrates several components of a system, according to some embodiments.

### DETAILED DESCRIPTION OF THE INVENTION

Various embodiments provide for extracting substances from a suspension. Suspended particles may include an interior having at least a portion of a first polarity (e.g., having a substantially nonpolar moiety) that may be separated from the liquid in which the particles are suspended (e.g., a polar liquid) by an exterior having a polarity that more closely matches that of the liquid. Such an exterior may inhibit or prevent interaction of a portion of the interiors of the particles with a solvent (added to the liquid) having a matching polarity. Disrupting the exteriors to expose the interiors to the solvent may enable agglomeration of various portions of the interiors with other components having similar polarity. For example, intracellular water may agglomerate with the aqueous liquid of the suspension, and nonpolar portions of the interiors (e.g., lipids) may agglomerate with other nonpolar portions and/or a nonpolar solvent. Agglomeration may include coalescing of non-polar portions with non-polar portions from other particles, and/or coalescing of polar portions (e.g., of the interiors) with likewise polar components of the suspension. Agglomeration may reduce a ratio of surface area to volume of a substance, which may ease separation of the substance.

In some embodiments, exterior layers or coatings may be disrupted or otherwise breached in the presence of a solvent having a polarity that more closely matches that of at least a portion of the interiors. The solvent may be immiscible in the liquid and have a polarity that dissolves or matches at least a portion of the interiors of the suspended particles.

For example, an aqueous suspension of cells may include interior lipids that are substantially nonpolar. The lipids may be surrounded and/or otherwise enclosed by substantially polar exteriors (e.g., cell walls). The lipids might be soluble in a nonpolar solvent (e.g., added to the suspension), but may be prevented from interacting with the nonpolar solvent by the polar exteriors. Extraction of the lipids may include disrupting or otherwise “opening” the exteriors in the presence of a substantially nonpolar solvent, allowing contact between the solvent and at least a portion of the interiors.

Various embodiments are described for cases in which particles are suspended in a polar liquid, and nonpolar interior portions are separated from the polar liquid by polar exteriors. Other embodiments may be directed toward a nonpolar liquid suspending particles having polar interiors (or portions) and an exterior of the particles may be nonpolar (e.g., reverse micelles).

A suspension may comprise a suspended phase and a liquid (e.g., water, seawater, growth media, and/or other liquid phases). A suspended phase may be a solid, a liquid, a composite, or another phase. Suspended phases may include small particles (e.g., less than a few mm, less than 1 mm, less than 100 microns, less than 10 microns, less than 1 micron, or even less than 100 nm). A suspended phase may include a portion having a polarity, moiety, or chemical affinity similar to that of the liquid (e.g., a miscible portion). A suspended phase may include a portion having a polarity, moiety, or chemical affinity that is substantially different than that of the liquid (e.g., an immiscible portion).

A suspended phase may include one or more photosynthetic organisms, such as algae, diatoms, bacteria, and the like. Some photosynthetic organisms include lipids, proteins, carbohydrates, and other biomass. Certain photosynthetic organisms include frustules. Some suspensions include aqueous liquids (e.g., water, natural seawater, synthetic seawater, and the like). Some suspensions have a concentration by mass of suspended phase in the liquid below 90%, below 80%, below 60%, below 40%, below 20%, below 10%, below 5%, below 2%, or even below 1%.

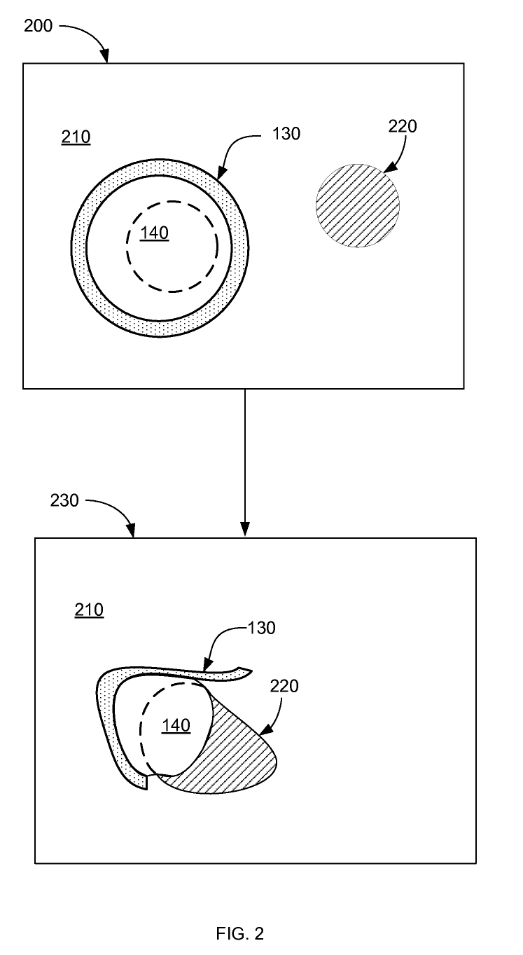

FIG. 2 illustrates disruption of an exterior layer, according to some embodiments. In a first state 200, a polar aqueous liquid 210 may suspend particles with interiors having substantially a nonpolar portion 140 (e.g., a lipid) surrounded by a substantially polar exterior 130. A nonpolar solvent 220 may be immiscible with aqueous liquid 210, but may dissolve nonpolar portion 140. Aqueous liquid 210 and polar exterior 130 separate nonpolar solvent 220 and nonpolar portion 140, typically preventing their interaction.

Various aspects provide for disrupting exterior layers (e.g., exterior 130). In second state 230, exterior 130 has been disrupted (e.g., removed, damaged, rearranged, or otherwise breached) to allow interaction between nonpolar solvent 220 and nonpolar portion 140. This interaction may result in dissolution of nonpolar portion 140 into nonpolar solvent 220. With the polar exterior disrupted, these discrete nonpolar solutions may agglomerate. Agglomerated volumes may be segregated (e.g., using gravity, centrifuging, filtration, and the like) and extracted.

Extracting may include segregating a suspension into segregation products. A suspension may be segregated into two, three, four, five, or more segregation products. Some suspensions may be segregated by density, with higher density components (e.g., water) below lower density components (e.g., lipids and/or nonpolar solvents).

A lipid-containing product may include a substantial fraction of a nonpolar solvent. In some embodiments, the intrinsic liquid component of the suspension (e.g., water) is preferentially segregated into a segregation product. A segregation product may have a majority of and/or be predominantly composed of a certain component (e.g., a lipid, water, non-lipid biomass, and the like). “Predominantly composed of” may include over 50%, over 70%, over 90%, over 95%, or even over 99%.

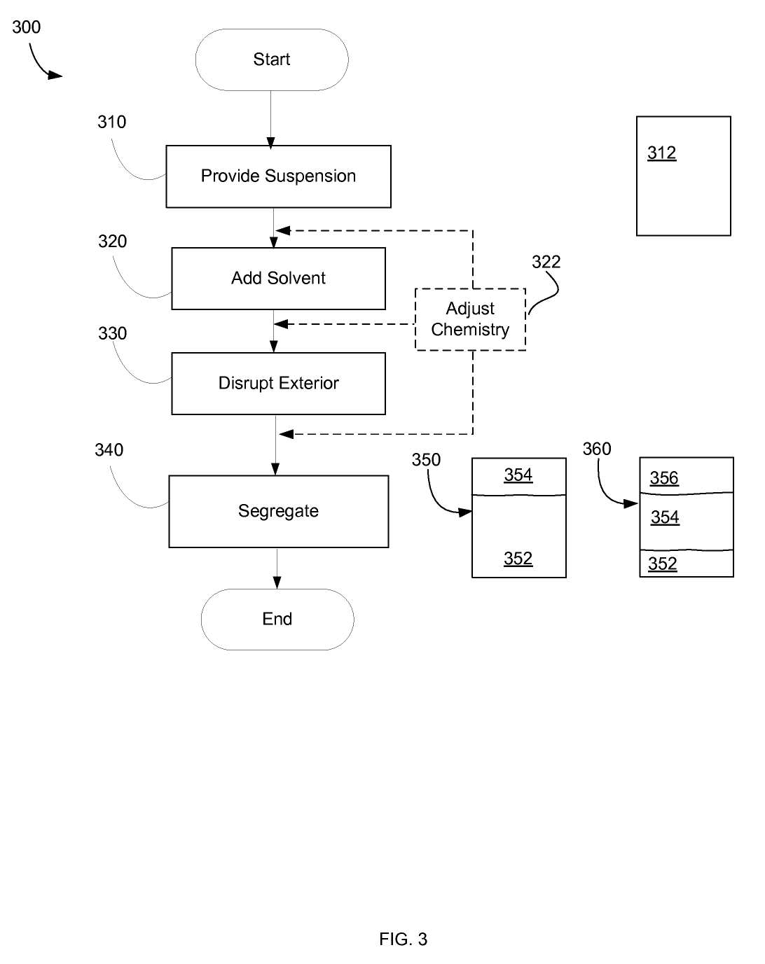

FIG. 3 illustrates a method according to some embodiments. Exemplary method 300 includes steps 310, 320, 330, and 340, and optional step 322. A suspension is provided in step 310 (e.g., suspension 312, illustrated to the right of step 310). The suspension may include a polar and/or aqueous liquid (e.g., water, seawater, synthetic seawater, brackish water, and/or growth media) in an amount greater than 10%, 20%, 50%, 70%, 90%, or even 95% by mass as compared to the suspended phase. In some cases, the suspended phase may be less than 5%, or even less than 2% of the suspension. Some suspensions include algae and/or diatoms suspended in (natural or synthetic) seawater, and may include various components of biomass such as lipids, proteins, carbohydrates, cellulose, and the like. The suspended phase may include particles having an interior (e.g., with a nonpolar portion) and an exterior (e.g., a polar exterior).

A nonpolar solvent may be added in step 320. In some cases, the solvent includes one or more alkanes (e.g., pentane, heptane, and/or hexane), esters, aldehydes, ethers, furans (e.g., THF), and/or ketones (e.g., MEK), halocarbons (e.g., TCE), and/or mixtures thereof. A nonpolar solvent may include a solvent that is at least partially nonpolar (e.g., various alcohols and/or acetone). A solvent may have a density below 1 g/cc, below 0.9 g/cc, below 0.8 g/cc, or even below 0.7 g/cc. Certain compounds (e.g., lipids) may dissolve in a nonpolar solvent. An added solvent may be sufficiently nonpolar that agglomeration of the solvent and solute is rapid (e.g., the nonpolar and polar volumes “phase separate”). A nonpolar solvent may have a dielectric constant below 10. A nonpolar solvent may have a solubility limit below 1% in the aqueous liquid. A nonpolar solvent with dissolved lipids may have a lower density than the aqueous liquid.

The solvent may be added in an amount similar to the amount of the suspended biomass that interacts with the nonpolar solvent. The solvent may be added in an amount similar to the total amount of suspension (e.g., liquid and solids). For some suspensions, an amount of nonpolar solvent may be approximately 1, 3, 5, 10, or even 20 times the amount of suspended biomass. In some embodiments, suspended biomass may be approximately two percent of the liquid (by mass), and an amount of nonpolar solvent may be less than 50%, 20%, 10%, 1%, or even less than 0.1% of the liquid.

Exteriors of the suspended particles may be disrupted in step 330. Disruption may include physical disruption (e.g., shearing the particles), chemical disruption, and/or thermal disruption (e.g., via a thermotropic phase transition). Disruption may include various chemical processes (e.g., lysing) to disrupt cell walls and/or otherwise change the chemistry of the suspension or components of the suspension. A surfactant may be added. Disruption may breach the exterior layers of the particles and/or otherwise allow contact between the interiors of the particles and the solvent.

Disruption may include agitation, which may include a range of applications of physical energy to the suspension. Agitation may include the application of localized mechanical forces (e.g., shear, tension, and/or compression). Agitation may include shaking, vibrating, ultrasonicating, megasonicating, and other applications of oscillatory energy. Agitation may include stirring (e.g., mixing at 50 rpm, 100 rpm, or even 500 rpm). Agitation may include injecting liquids and/or gases (e.g., with jets), stirring, gas bubble precipitation, and or other mixing (e.g., with impellers, blades, and the like). Agitation may include milling (e.g., ball milling). Agitation may disrupt suspended diatoms, (e.g., fracture frustules), which may expose biomass within the frustules (e.g., to the solvent) and may increase the surface area of the suspended phase exposed to liquid and/or solvent.

Temperature may be controlled (e.g., to 25° C., 75° C., 100° C., 150° C., 200° C., or even higher). Pressure may be controlled (e.g., by performing various steps in a pressure vessel). In some cases, a pressure is maintained at a sufficiently high level (e.g., 10 psi, 50 psi, 100 psi, or even 500 psi) that the liquid and/or added nonpolar solvent remains liquid at temperatures above its boiling point at atmospheric pressure. Pressure and temperature may be controlled to create a supercritical fluid (e.g., supercritical CO₂).

Chemistry may be adjusted at one or more points in the process (e.g., chemistry of the suspension, the mixture of suspension and solvent, the disrupted mixture, during segregation, and/or chemistry of one or more segregation products). In exemplary FIG. 3, chemistry is adjusted at optional step 322. Various chemicals may be added before, during, and/or after disruption and/or segregation. Acids, bases, and the like may be added, and may be used to modify pH of the suspension, mixture, or a product. In some cases, segregation of a substance to a segregation product may be modified via chemical addition. For example, Iron may be present in non-lipid biomass. Decreasing pH (e.g., with an acid such as Hydrochloric Acid) may increase the solubility of the Iron, which may result in dissolution of the Iron (e.g., into the aqueous liquid). Addition of a base (e.g., a hydroxide) may increase pH, and may cause precipitation of a substance, which may improve segregation and/or improve extraction of a segregation product.

In some embodiments, the addition of chemicals may reduce the overall energy requirements of an extraction process (e.g., by reducing a temperature of disruption, or a force needed for segregation). The speed (e.g., of segregation) may be increased for some processes via chemical addition (e.g., pH adjustment). An addition of ammonia to the suspension may increase the solubility of proteins in a polar liquid.

Steps 320 and 330 may be performed in any order or at the same time. Disruption may result in agglomeration of self-similar portions of the “interiors” of the suspended particles (often dissolved in the solvent), forming larger volumes (e.g., having larger volume to surface ratio) that may be separated or segregated.

The mixture may be segregated in step 340. Segregation may separate the suspension into one or more segregation products. Segregation may include allowing the suspension to segregate using gravity (e.g., heavier products at the bottom and lighter products at the top). Segregation may be enhanced by centrifuging, for example at greater than 10×, 100×, 1000×, or even 10,000×, the force of gravity (RCF). Segregation may include causing at least a portion of the mixture to pass through a membrane or filter that is permeable to a first substance and substantially impermeable to a second substance. Membranes or filters may have mean pore sizes ranging from 1 nm to 300 microns, or between 100 nm and 10 microns. Some membranes or filters may have a mean pore size between 10 microns and 1 mm.

A membrane or filter may be fabricated from a variety of porous and/or semi-permeable materials, such as stainless steel, aluminum, alumina, silicon carbide, zirconia, glasses, woven materials (e.g., fiberglass), felted materials, polymers (e.g., polypropylene, polytetrafluoroethylene, expanded polytetrafluoroethylene, fluorinated polypropylene, carbon fiber, kevlar, and/or composite materials).

A system including a membrane may include a pressure vessel and associated apparatus to generate a pressure gradient (or other chemical potential gradient) across the membrane or filter (e.g., a pressure up to 30 bar, or a pressure between 2 bar and 100 bar). A membrane system may include an anti-fouling apparatus, a scraper to remove material from an inlet side of the membrane, and/or other apparatus. A membrane system may incorporate tangential flow of the mixture or suspension. A membrane may be vibrated.

Two exemplary segregations are illustrated to the right of step 340. Segregated suspension 350 includes first segregation product 352 (e.g., aqueous liquid and intracellular water) and second segregation product 354 (e.g., lipids and nonpolar solvent). Segregated suspension 360 includes first segregation product 352 (e.g., polar liquids), second segregation product 354 (e.g., a slurry of proteins and carbohydrates), and third segregation product 356 (e.g., nonpolar solvent and dissolved lipids).

In some embodiments, density differences between nonpolar solvents, lipids, non-lipid biomass, and water may result in a segregation of water at the bottom and nonpolar solvent/lipids or non-lipid biomass at the top of the container. In some cases, non-lipid biomass (e.g., proteins, carbohydrates, fiber, and/or other substances) may be present in a middle layer.

Typically, a segregated mixture has segregation products with properties (e.g., concentration of a component) appropriate for subsequent processing and/or use. Segregation products may be removed. In some cases, it may be convenient to remove a top product (e.g., by decanting a product comprising lipids and nonpolar solvents). In some cases, it may be convenient to remove a bottom product (e.g., aspirating the product through a needle). A majority product may be removed (e.g., for segregated suspensions in which water is the largest component, it may be convenient to remove water first). A first product may be removed, the remaining mixture segregated or re-segregated, and a second product may be removed.

A segregation product may contain a majority of a substance (e.g., a majority of the lipids). In some cases, a segregation product may contain over 70% or even over 90% of a substance. Small amounts of a minority substance may be present in a segregation product. For example, a segregation product having the majority of the aqueous liquid may include a few percent of a nonpolar solvent. A segregation product including most of the non-lipid biomass may include some liquid.

A segregation product may include a majority of two or more substances (i.e., a first product may contain more of both a first substance and a second substance than does a second product). For example, a segregation product may include a majority of the lipids and a majority of the nonpolar solvent, or a segregation product may include a majority of the proteins and a majority of the carbohydrates.

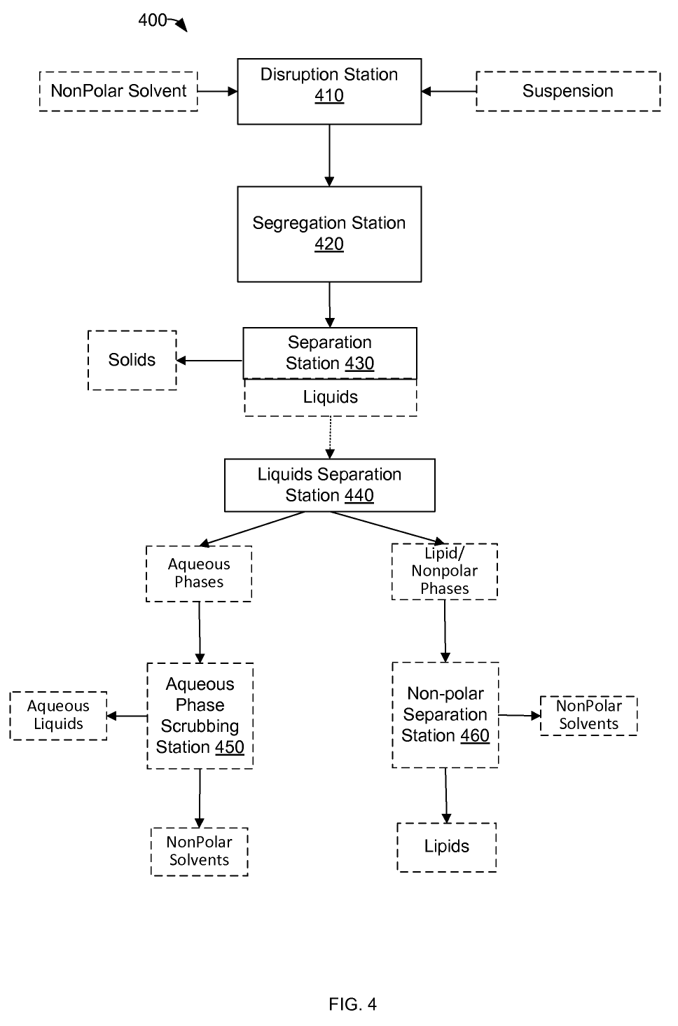

FIG. 4 illustrates several components of a system, according to some embodiments. In some embodiments, system 400 includes a disruption station 410, a segregation station 420, a separation station 430, and a liquids separation station 440. Some embodiments may include an aqueous phase scrubbing system 450, and a nonpolar separation station 460. Some components may be omitted; some components may be combined.

In some embodiments, disruption station 410 receives a suspension and solvent (e.g., a nonpolar solvent for an aqueous liquid suspension). Disruption station 410 may disrupt the exteriors of suspended particles. Disruption station 410 may include a mixer, a low shear mixer, a high shear mixer, and the like. In some embodiments, disruption station 410 includes an ultrasonication apparatus and means to inject nonpolar solvent into the suspension (e.g., an inlet tube, a valve, a port, and the like). Disruption station 410 may include a vortexer (e.g., a blender). An agent to enhance lysing (e.g., chalk, sand, gypsum, and the like) may be provided.

Disruption of the particles may result in contact between the interiors and the solvent. The mixture of solvent, liquid, and disrupted particles may pass to the segregation station 420, which segregates the mixture. In some embodiments, segregation station 420 includes a centrifuge, a decanter, a membrane, and/or a filter. A segregated “suspension” typically includes two or more segregation products. For illustrative purposes, FIG. 4 describes facilities directed toward a mixture that may be segregated into a solids-containing segregation product (e.g., containing cell walls, cellulose, and the like) and a liquids-based segregation product. A nonpolar liquid segregation product and an aqueous liquid segregation product may be created.

Solids separation station 430 may remove one or more products containing the majority of the solids. Liquids may be separated in liquids separation station 440, which may be integrated with solids separation station 430 in some embodiments. Liquids separation station 440 may separate liquids into substantially polar liquids and substantially nonpolar liquids. In some embodiments, lipids may be dissolved into the nonpolar liquids. For some suspensions, aqueous and nonpolar liquids may be segregated by their density differences using centrifuging, and separated using aspiration and/or decanting.

Various segregation products may include minority phases. For example, a solids-containing product may include some liquid (e.g., an aqueous slurry of cell walls). An aqueous segregation product may include some nonpolar solvent, and a segregation product substantially based on the nonpolar solvent may contain some water (e.g., trace amounts, or even a few percent). In some cases, minority phases may be retained with the majority phase (e.g., water may be retained with proteinaceous and/or siliceous phases to form a slurry). In some cases, minority phases may be removed.

Aqueous phase scrubbing station 450 may remove residual nonpolar solvents from the aqueous liquids (e.g., using distillation, such as flash distillation). Nonpolar separation station 460 may separate nonpolar liquids (e.g., lipids) from nonpolar solvents, and may also separate different nonpolar solvents. Nonpolar separation station 460 may include one or more distillation apparatus. Nonpolar separation station 460 may include a cooling apparatus to solidify a portion of the nonpolar phases (e.g., solidify the lipids), which may be removed via solid removal methods (e.g., filtration).

In some embodiments, a segregation product is “recycled” through another segregation step (e.g., to increase “purity”). A segregation product may be recycled into other parts of the system. For example, extracted aqueous liquid may be used to grow additional algae, or extracted nonpolar solvent may be recycled for use in disruption station 410.

While various aspects have been described in the context of an aqueous suspension to which a nonpolar solvent may be added, certain embodiments may be directed toward suspensions based on a nonpolar liquid to which a substantially polar solvent may be added.

Some embodiments include sensors to sense various parameters (e.g., concentration, depth, photosynthetic rate, clarity, pH, mass, dielectric constant, transparency, opacity, temperature, pressure, and other characteristics). Apparatus may monitor various sensors, and systems may be actuated by automated controls (solenoid, pneumatic, piezoelectric, and the like). Some embodiments include a computer readable storage medium coupled to a processor and memory. Executable instructions stored on the computer readable storage medium may be executed by the processor to perform various methods described herein. Sensors and actuators may be coupled to the processor, providing input and receiving instructions associated with various methods. Certain instructions provide for closed-loop control of various parameters via coupled sensors providing input and coupled actuators receiving instructions to adjust parameters.

Certain embodiments include materials. A biofuel may be synthesized from a lipid extracted according to various methods and/or a carbohydrate extracted according to various methods. A particulate biomass material (e.g., defatted cellular material) may be synthesized according to various methods. Particle sizes may be below 1 mm, 100 microns, 10 microns, or even below 1 micron. In some cases, particle sizes are governed by a size of the exterior layer (e.g., a cell wall) from which a particulate material is fabricated. Various embodiments include nutrients (e.g., proteins, carbohydrates, fats), which may be extracted according to certain methods.

The above description is illustrative and not restrictive. Many variations of the invention will become apparent to those of skill in the art upon review of this disclosure. The scope of the invention should, therefore, be determined not with reference to the above description, but instead should be determined with reference to the appended claims along with their full scope of equivalents.

What is claimed is:

1. A method for extracting suspended lipids from an aqueous suspension, the method comprising:
providing a suspension including an aqueous liquid and suspended particles, the suspended particles including lipids substantially contained within polar exterior layers;
adding a nonpolar solvent to the suspension to form a mixture;
disrupting the exterior layers to expose the lipids to the nonpolar solvent; and
segregating the mixture into first and second products, the first product having a higher amount of the lipids than the second product.

2. The method of claim 1, wherein a concentration of the aqueous liquid in the suspension is above 10% by mass.

3. The method of claim 2, wherein the concentration is above 50% by mass.

4. The method of claim 3, wherein the concentration is above 90% by mass.

5. The method of claim 1, wherein the lipids include one or more triglycerides.

6. The method of claim 1, wherein the exterior layers include cell walls.

7. The method of claim 1, wherein the lipids include hydrophilic moieties, and the exterior layers result from an exterior configuration of the hydrophilic moieties.

8. The method of claim 1, wherein the aqueous liquid includes natural or synthetic seawater.

9. The method of claim 1, wherein the nonpolar solvent includes an alkane.

10. The method of claim 9, wherein the alkane includes hexane.

11. The method of claim 1, wherein disrupting includes agitating the suspension or mixture.

12. The method of claim 11, wherein agitating includes stirring.

13. The method of claim 11, wherein agitating includes ultrasonicating.

14. The method of claim 1, wherein the particles include cells, and disrupting includes lysing the cells.

15. The method of claim 1, wherein disrupting includes heating the mixture.

16. The method of claim 15, wherein heating includes heating to above 80 degrees Celsius.

17. The method of claim 16, wherein heating includes heating to above 150 degrees Celsius.

18. The method of claim 15, wherein heating comprises heating the mixture to a temperature above a boiling point at ambient pressure of the nonpolar solvent or the aqueous liquid.

19. The method of claim 18, further comprising controlling a pressure above the heated mixture to a value that prevents boiling of the nonpolar solvent and the aqueous liquid.

20. The method of claim 19, wherein the controlled pressure is below 500 psig.

21. The method of claim 20, wherein the controlled pressure is below 200 psig.

22. The method of claim 1, wherein segregating includes allowing the mixture to settle according to gravity.

23. The method of claim 1, wherein segregating includes centrifuging.

24. The method of claim 23, wherein the centrifuging induces an acceleration that does not exceed 500 times the force of gravity.

25. The method of claim 23, wherein the centrifuging induces an acceleration that does not exceed 3000 times the force of gravity.

26. The method of claim 23, wherein the centrifuging induces an acceleration between 5 times and 50,000 times the force of gravity.

27. The method of claim 23, wherein the centrifuging induces an acceleration between 10 times and 10,000 times the force of gravity.

28. The method of claim 1, wherein the first product includes a larger portion of the nonpolar solvent than does the second product.

29. The method of claim 1, wherein the segregated mixture further comprises a third product having a majority of one or more non-lipid biomass substances.

30. A material comprising at least one of the non-lipid biomass substances fabricated according to claim 29.

31. The method of claim 1, wherein the particles include algae.

32. The method of claim 31, wherein at least a portion of the algae include a member of the genus Nannochloropsis.

33. The method of claim 1, wherein the particles include diatoms.

34. The method of claim 1, wherein the second product includes a majority of the aqueous liquid.

35. The method of claim 1, wherein segregating includes segregating the mixture into first, second, and third products.

36. The method of claim 35, wherein the first product includes a majority of the lipids, the second product includes a majority of a non-lipid biomass, and the third product includes a majority of the aqueous liquid.

37. The method of claim 36, wherein the non-lipid biomass includes one or more proteins.

38. The method of claim 36, wherein the non-lipid biomass includes one or more carbohydrates.

39. A lipid extracted from an aqueous suspension using a method according to claim 1.

40. A biofuel synthesized from a substance extracted from an aqueous suspension using a method according to claim 1.

41. The biofuel of claim 40, wherein the extracted substance includes a lipid, and the biofuel is synthesized from the lipid using a process that includes transesterification of the lipid.

42. The method of claim 1, wherein segregating includes forcing the mixture through a membrane or filter that is permeable to at least a portion of one of the first and second products and substantially impermeable to at least a portion of the other of the first and second products.

43. The method of claim 42, wherein the membrane or filter has a mean pore size between 1 nm and 1 mm.

44. The method of claim 43, wherein the membrane or filter has a mean pore size between 1 micron and 100 microns.

45. The method of claim 42, wherein forcing includes applying a pressure that does not exceed 30 bar to the mixture.

46. The method of claim 42, wherein forcing includes applying a pressure between 5 bar and 100 bar to the mixture.

47. The method of claim 1, further comprising adjusting the chemistry of any of the suspension, mixture, and products.

48. The method of claim 47, wherein adjusting the chemistry includes adjusting pH.

49. The method of claim 47, wherein adjusting the chemistry includes adding one or more acids.

50. The method of claim 47, wherein adjusting the chemistry includes adding one or more bases.

51. The method of claim 47, wherein adjusting the chemistry includes dissolving a chemical species from a gas phase passed through any of the suspension, mixture, and products.

52. A method comprising:
providing a suspension in a pressure vessel, the suspension including suspended cells in natural or synthetic seawater, the cells including lipids substantially contained within polar exterior layers;
adding hexane to the suspension to form a mixture;
heating the mixture to a temperature between 20 and 200 degrees Celsius;
controlling a pressure above the heated mixture to a value that prevents boiling of the hexane or seawater;
agitating the heated mixture to disrupt the exterior layers; and
segregating the agitated mixture into first and second products, the first product having a majority of the lipids and the second product having a majority of the seawater.

53. A particulate material fabricated according to a method comprising:
providing a suspension including an aqueous liquid and suspended cells, the suspended cells having a mean size below 100 microns and including lipids substantially contained within walls of the cells;
adding a nonpolar solvent to the suspension to form a mixture;
disrupting the cell walls to expose the lipids to the nonpolar solvent;
segregating the mixture into first, second, and third products, the first product containing a majority of the cell walls, the second product containing a majority of the lipids, and the third product containing a majority of the aqueous liquid; and
removing the first product.

54. A system for extracting substances from a suspension, the system comprising:
a disruption station having a first inlet to receive the suspension, the suspension including:
a liquid, and
suspended particles having exterior layers that are chemically compatible with the liquid and interiors having at least a portion that is substantially immiscible with the liquid,
the disruption station having a second inlet to receive a solvent that dissolves the portions of the interiors that are immiscible with the liquid, the disruption station configured to disrupt the exterior layers; and
a segregation station configured to segregate the disrupted suspension into at least first and second segregation products, the first segregation product including a greater amount of the solvent and dissolved portions than the second segregation product.

55. The system of claim 54, further comprising a separation station configured to remove at least one of the segregation products.

56. The system of claim 54, wherein the disruption station includes a pressure vessel.

57. The system of claim 54, wherein the disruption station may be heated to a temperature above 30 degrees Celsius.

58. The system of claim 54, wherein the segregation station includes a centrifuge.

59. The system of claim 54, wherein the segregation station includes:
a membrane or filter that is permeable to a first substance in the disrupted suspension and substantially impermeable to a second substance in the disrupted suspension; and
a pressure vessel configured to force the first substance through the membrane.
